(19) United States
(12) Patent Application Publication
(10) Pub. No.: US 2021/0353494 A1
(43) Pub. Date: Nov. 18, 2021

(54) ASSIST DEVICE

(71) Applicant: JTEKT CORPORATION, Osaka-shi (JP)

(72) Inventors: Yoshitaka YOSHIMI, Kashiba-shi (JP); Kazuyoshi OHTSUBO, Chiryu-shi (JP); Terukazu SENDA, Obu-shi (JP)

(73) Assignee: JTEKT CORPORATION, Osaka-shi (JP)

(21) Appl. No.: 17/319,341

(22) Filed: May 13, 2021

(30) Foreign Application Priority Data
May 18, 2020 (JP) 2020-086987

(51) Int. Cl. A61H 1/02

(52) U.S. Cl. CPC A61H 1/0292; A61H 1/0244; A61H 2201/149; A61H 2201/1616; A61H 2201/1621; A61H 2201/163; A61H 2201/1626; A61H 2201/1652; A61H 2201/5025; A61H 2201/5097; A61H 2201/1215; A61H 2201/50; A61H 2201/1642

(57) ABSTRACT

An assist device includes a first body-worn unit, a second body-worn unit, a belt body, an actuator, and a controller. The actuator includes a pulley and a motor that causes the pulley to reel and unreel a part of the belt body. The controller is configured to execute a mode switching process of switching a control mode of the motor to one a restriction mode in which a maximum rotation speed of the motor is restricted to a predetermined rotation speed and a normal mode in which the motor is operated within a rotation speed range higher than the predetermined rotation speed.

### CROSS-REFERENCE TO RELATED APPLICATION

This application claims priority to Japanese Patent Application No. 2020-086987 filed on May 18, 2020, incorporated herein by reference in its entirety.

### BACKGROUND

### 1. Technical Field

The disclosure disclosed herein relates to an assist device.

### 2. Description of Related Art

Various assist devices that are worn on the bodies of users (persons) to assist the users in tasks have been proposed. When lifting a heavy object, for example, a user of an assist device can perform the task with a smaller force (with less burden). Such an assist device is disclosed, for example, in Japanese Patent Application Publication No. 2018-199205 (JP 2018-199205 A).

### SUMMARY

The assist device disclosed in JP 2018-199205 A has a frame made of metal etc. that is worn by a user. An output of an actuator installed in this frame is transmitted to the upper body and the lower body of the user through a link mechanism. This assists the user, for example, in an action of lifting a heavy object.

Other than physically taxing actions such as lifting a heavy object, actions in which users need assistance include, for example, actions of assisting (helping) a person such as a sick person or an elderly person in performing activities of daily living. When a user performs a physically taxing task, a high-output assist device as disclosed in JP 2018-199205 A is effective.

However, when a user assists a person such as a sick person or an elderly person, the performance of a high-output assist device is in some cases superfluous. In addition, a high-output assist device uses many rigid members, including a link mechanism and a frame made of metal etc., and has a massive structure to produce a high output. Thus, the assist device is heavy and the rigid members restrict the movement of the user.

As a solution, the inventor of the present disclosure has already proposed an assist device that is lightweight and comfortable to wear (e.g., Japanese Patent Application No. 2019-043462). This assist device includes a first body-worn unit that is worn on the shoulders of a user, a second body-worn unit that is worn on the left and right legs of the user, a belt body that is provided along the back side of the user, across the first body-worn unit and the second body-worn unit, and an actuator. The actuator is provided in the first body-worn unit and allows a part of the belt body to be reeled and unreeled.

As the actuator reels a part of the belt body, a tensile force is exerted on the belt body. This tensile force acts on the user as an assist force. Thus, the burden on the user is reduced, for example, when the user assists a person as described above.

The actuator includes a pulley around which the belt body is wound, and a motor that drives the pulley to cause the pulley to reel and unreel a part of the belt body. When the motor is put under current control, the rotation speed of the motor is not always taken into account. Thus, the motor can momentarily reach an excessively high rotation speed. Since the kinetic energy of rotational motion increases in proportion to the square of the rotation speed of the motor, the noise of the motor may become problematic when the motor reaches an excessively high rotation speed.

An aspect of the present disclosure is an assist device. This assist device includes: a first body-worn unit that is worn on at least one of the shoulders and the chest of a user; a second body-worn unit that is worn on at least one of left and right legs and a waist of the user; a belt body that is provided along the back side of the user, across the first body-worn unit and the second body-worn unit; an actuator that is provided in one of the first body-worn unit and the second body-worn unit and configured to reel and unreel a part of the belt body; and a controller configured to control the actuator. The actuator includes a pulley around which a part of the belt body is wound, and a motor that causes the pulley to reel and unreel a part of the belt body. The controller is configured to execute a mode switching process of switching a control mode of the motor to one of a restriction mode in which a maximum rotation speed of the motor is restricted to a predetermined rotation speed and a normal mode in which the motor is operated within a rotation speed range higher than the predetermined rotation speed.

With this configuration, in the restriction mode, the maximum rotation speed of the motor is restricted to a predetermined rotation speed lower than that in the normal mode, and therefore noise made by the motor as it rotates can be reduced compared with that in the normal mode. Thus, the noise of the motor can be reduced by switching the control mode of the motor.

Usually, it is at night that the noise of the motor is problematic. Therefore, in the above assist device, the controller may be configured to execute the mode switching process according to the current time. This configuration allows the user to reduce the noise of the motor according to the current time by, for example, switching the control mode of the motor to the restriction mode during the nighttime and switching the control mode of the motor to the normal mode at other times of the day than the nighttime.

The above assist device may further include an input part configured to receive an external input. The controller may be configured to execute the mode switching process based on the external input. With this configuration, the user of the assist device etc. can cause the controller to execute the mode switching process by making an external input into the input part. Thus, the user can reduce the noise of the motor as necessary.

These configurations can reduce the noise of the motor.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a back view showing one example of an assist device;

FIG. 2 is a back view of the assist device attached to the body of a user;

FIG. 3 is a side view of the assist device attached to the body of the user;

FIG. 4 is a view illustrating a state where the user wearing the assist device has assumed a forward leaning posture;

FIG. 5 is a view illustrating a control box and a belt body;

FIG. 6 is a block diagram showing a control configuration of the assist device;

FIG. 7 is a view illustrating a case where the user wearing the assist device changes his or her posture;

FIG. 8 is a flowchart showing one example of a mode switching process; and

FIG. 9 is a block diagram showing a configuration around a controller according to another embodiment.

### DETAILED DESCRIPTION OF EMBODIMENTS

Overall Configuration of Assist Device 10

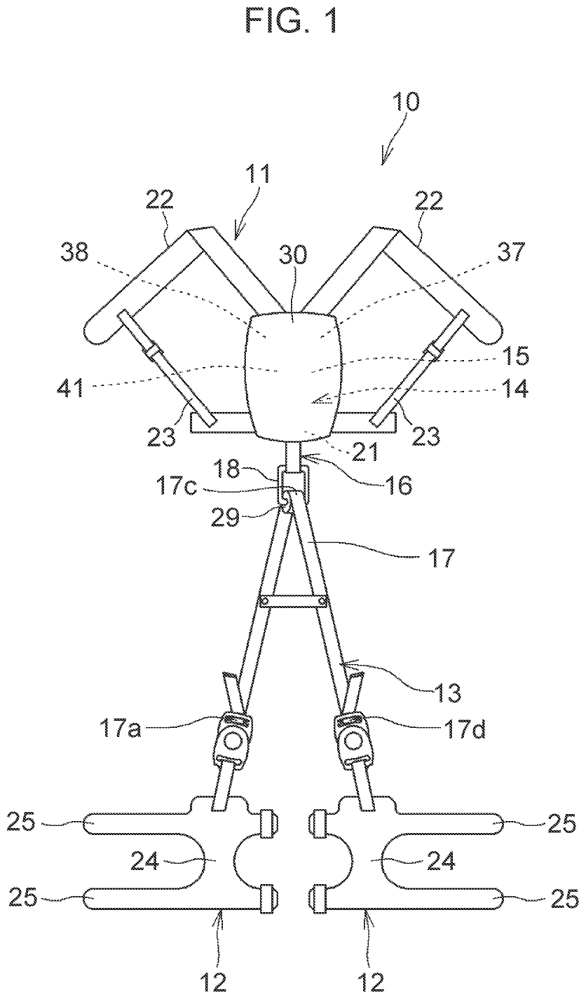

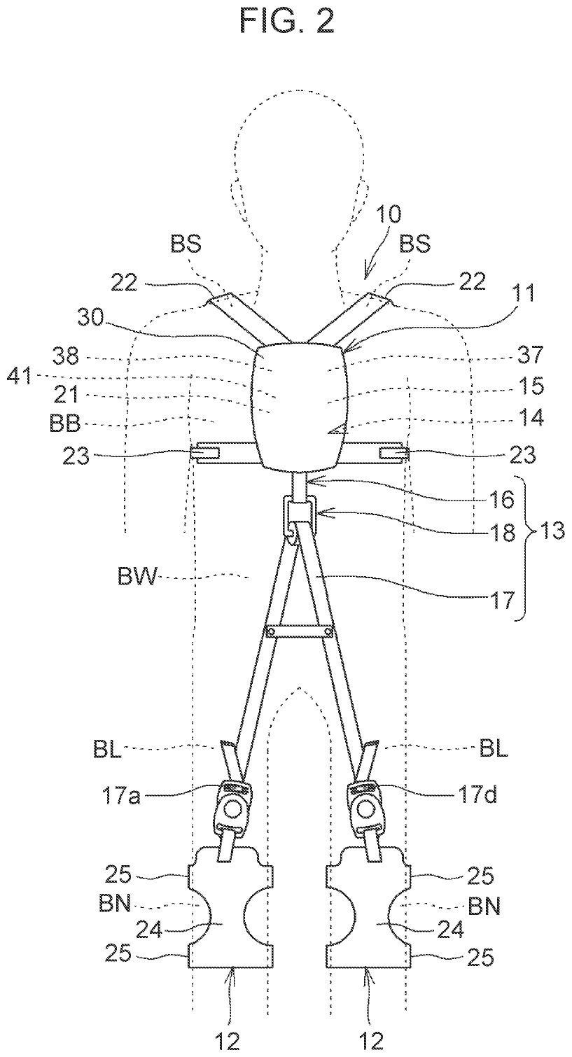

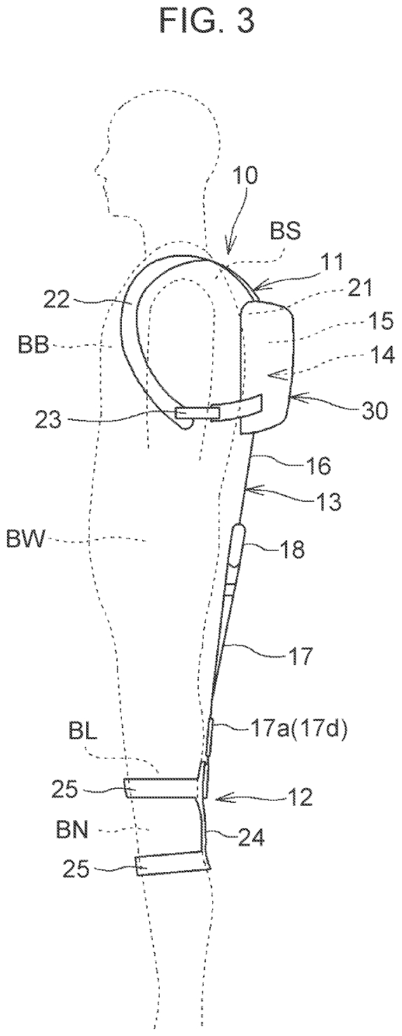

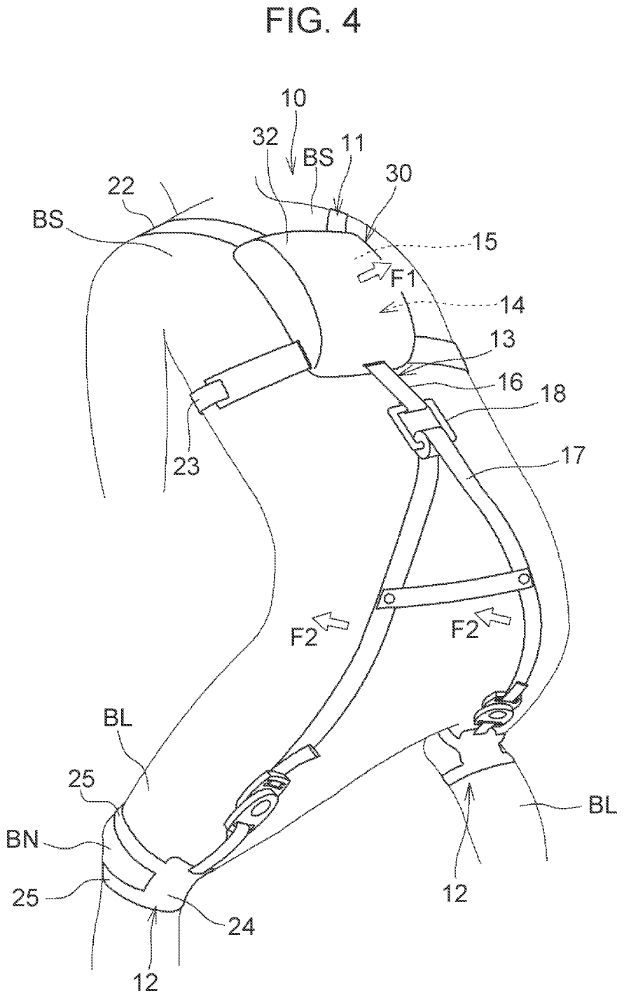

FIG. 1 is a back view showing one example of an assist device. FIG. 2 is a back view of the assist device attached to the body of a user. FIG. 3 is a side view of the assist device attached to the body of the user. FIG. 4 is a view illustrating a state where the user wearing the assist device has assumed a forward leaning posture (forward bending posture). An assist device 10 shown in FIG. 1 includes one first body-worn unit 11 that is worn on the left and right shoulders BS that are parts of the body of the user (person), and two second body-worn units 12 that are worn on the left and right legs BL that are other parts of the body of the user. The first body-worn unit 11 can be worn on at least either the shoulders BS or the chest BB of the user, and may have a form different from that shown in the drawings. In this disclosure, the second body-worn units 12 are worn on the knees BN in the legs BL. The second body-worn units 12 may also have a form different from that shown in the drawings.

In the assist device 10 of this disclosure, left and right sides are left and right sides of a user wearing the assist device 10 and in an upright standing posture. Front and back sides are front and back sides of the user, and upper and lower sides are upper and lower sides of the user. The upper side is the side of the head of the user and the lower side is the side of the feet of the user.

Other than the first body-worn unit 11 and the left and right second body-worn units 12, the assist device 10 includes a belt body 13, an actuator 14, a controller 15, a battery 37, a sensor 38, and a communication device 41.

The first body-worn unit 11 is worn on the shoulders BS of the user. One of the second body-worn units 12 is worn on the left knee BN of the user. The other second body-worn unit 12 is worn on the right knee BN of the user. The left second body-worn unit 12 and the right second body-worn unit 12 are symmetrical and have the same configuration. The first body-worn unit 11 and the two second body-worn units 12 are worn at two sites, namely, on the shoulders BS and the legs BL, that are separated from each other on both sides of the waist BW that is a joint of the user.

The first body-worn unit 11 is made of flexible fabric or the like. The first body-worn unit 11 has a back main part 21 that is worn on the back of the user, and shoulder belts 22 and underarm belts 23 that are connected to the back main part 21. The back main part 21 is carried on the back of the user by the shoulder belts 22 and the underarm belts 23. Each underarm belt 23 connects the back main part 21 and the corresponding shoulder belt 22 to each other, and the length of each underarm belt 23 is adjustable. The lengths of the underarm belts 23 are adjusted so as to bring the back main part 21 into close contact with the back of the user. The first body-worn unit 11 is not movable in front-rear, left-right, and up-down directions relatively to the shoulders BS. The first body-worn unit 11 may include hard members, for example, as parts that are laid over the shoulders BS.

The second body-worn units 12 are made of flexible fabric or the like. Each second body-worn unit 12 has a knee main part 24 that is worn on the back side of the knee BN of the user, and knee belts 25 that are provided so as to extend from the knee main part 24. The knee belts 25 are wrapped around the knee BN at positions above and below the knee BN and fixed at their leading ends to the knee main part 24. The lengths of the knee belts 25 wrapped around the knee BN can be adjusted by means of an engaging member, such as a belt and a buckle or a touch-and-close fastener. These lengths are adjusted so as to bring the knee main part 24 into close contact with the back side of the knee BN. The second body-worn unit 12 is worn so as to be unable to move in the front-rear, left-right, and up-down directions relatively to the knee BN.

The belt body 13 is provided along the back side of the user so as to connect the first body-worn unit 11 and the second body-worn units 12 to each other. The belt body 13 has a first belt 16 that is provided on the upper body side, a second belt 17 that is provided on the lower body side, and a coupling member 18 that couples the first belt 16 and the second belt 17 together. Each of the first belt 16 and the second belt 17 is elongated and flexible. The coupling member 18 is made of metal and formed by a rectangular annular body called a flat ring. The coupling member 18 may be other than a flat ring, and may be a fastener such as a buckle. When the coupling member 18 is a buckle, the first belt 16 and the second belt 17 are respectively attached to upper and lower loops (hole) of the buckle. When the coupling member 18 is a buckle, it is highly convenient in that the second belt 17 can be separated from the first belt 16, for example, when cleaning the second belt 17. The coupling member 18 formed by a flat ring, a buckle, or the like may be made of resin other than metal.

Each of the first belt 16 and the second belt 17 is a band-like member made of fabric or leather and can curve along the shape of the body. Alternatively, each of the first belt 16 and the second belt 17 may be a string-like belt (wire-like member). Each of the first belt 16 and the second belt 17 of this disclosure is a non-stretchable member, i.e., has a property of hardly stretching or not stretching in a longitudinal direction thereof.

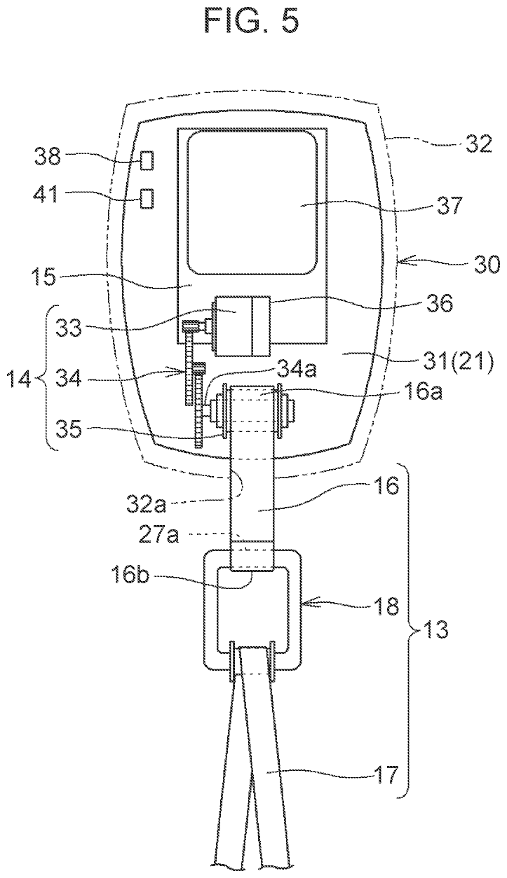

The assist device 10 of this disclosure includes a control box 30. The control box 30 is provided in the back main part 21 of the first body-worn unit 11. FIG. 5 is a view illustrating the control box 30 and the belt body 13. The control box 30 has a plate-shaped base 31 and a cover 32 that covers the base 31. To illustrate the internal structure of the control box 30, the cover 32 is indicated by an imaginary line (long dashed double-short dashed line) in FIG. 5. The base 31 may constitute the back main part 21 of the first body-worn unit 11.

The actuator 14, the controller 15, the battery 37, the sensor 38, the communication device 41, etc. are provided in a space formed between the base 31 and the cover 32. The cover 32 has an opening (cutout) 32a, through which the first belt 16 is passed.

The actuator 14 is provided inside the control box 30. In other words, the actuator 14 is provided in the first body-worn unit 11. The actuator 14 allows a part of the belt body 13 to be reeled and unreeled. For this purpose, the actuator 14 has a motor 33, a speed reducer part 34, and a driving pulley 35. The motor 33 is a brushless DC motor. The motor 33 can be rotated at a predetermined torque and a predetermined number of rotations based on a control command including a current command value that is output from the controller 15. The motor 33 is controlled so as to be able to rotate in normal and reverse directions based on the control command output from the controller 15. Thus, the motor 33 is put under current control by the controller 15.

Parameters relating to rotation of the motor 33, such as the rotation angle, the rotation speed, and the number of rotations, are detected by a rotation detector 36 that is mounted on the motor 33. The rotation detector 36 of this disclosure is a rotary encoder but may instead be a hall sensor or a resolver. A detection result of the rotation detector 36 is input into the controller 15. With the controller 15 controlling the operation of the motor 33 based on the detection result, the assist device 10 can generate an appropriate assist force.

The speed reducer part 34 is composed of a plurality of gears, and reduces the number of rotations of the motor 33 and rotates an output shaft 34a of the speed reducer part 34. The driving pulley 35 is coupled to the output shaft 34a, and these parts rotate integrally. One end part 16a of the first belt 16 is attached to the driving pulley 35. When the motor 33 rotates in the normal direction and causes the driving pulley 35 to rotate in one direction, the first belt 16 is reeled onto the driving pulley 35. When the motor 33 rotates in the reverse direction and causes the driving pulley 35 to rotate in the other direction, the first belt 16 is unreeled from the driving pulley 35.

Thus, the actuator 14 has the driving pulley 35 that can reel the belt body 13, and the motor 33 that causes the driving pulley 35 to perform an action of reeling the belt body 13. The first belt 16 is reeled and unreeled by the actuator 14.

The controller 15 is formed by a control unit including a microcomputer. The controller 15 has a function of controlling the operation of the actuator 14 (motor 33). As the sensor 38, a triaxial acceleration sensor is used. Signals of the sensor 38 are given to the controller 15. Based on the signals from the sensor 38, the controller 15 can infer the posture of the user. The battery 37 supplies electricity to the controller 15, the motor 33, the rotation detector 36, and the sensor 38. The sensor 38 may be provided outside the control box 30.

The communication device 41 is a device by which the assist device 10 communicates with a user terminal. The user terminal is a terminal device that the user uses to control the assist device 10 from outside.

The belt body 13 has the first belt 16, the second belt 17, and the coupling member 18 as described above. The one end part 16a of the first belt 16 is wound and fixed on the driving pulley 35. The other end part 16b of the first belt 16 is fixed on the coupling member 18. When the first belt 16 is reeled onto the driving pulley 35, the coupling member 18 is pulled up. When the coupling member 18 is forcibly pulled down, the first belt 16 is reeled out (pulled out) from the driving pulley 35. There is a correlation between the amount that the first belt 16 is reeled in or reeled out (pulled out) by the driving pulley 35 and the amount of rotation of an output shaft of the motor 33. A parameter relating to rotation of the motor 33 involved in reeling in or reeling out of the belt body 13 is detected by the rotation detector 36.

Sensor 38 and Controller 15

In FIG. 5, the sensor 38 is formed by a triaxial acceleration sensor as mentioned above. The controller 15 can execute various computation processes. An action and a posture of the user can be detected by the controller 15 performing computation processes on signals from the sensor 38. The sensor 38 has a configuration for outputting a signal according to the posture of the user, and functions as a posture detector that detects the posture of the user. Based on an output from the sensor 38, the controller 15 can detect a state, for example, whether the posture of the upper body of the user is in a forward leaning posture or an upright standing posture, or that the user has crouched.

There is a correlation between the amount that the belt body 13 is reeled onto and unreeled from the driving pulley 35 by the motor 33 and the posture of the user. Therefore, the controller 15 can infer the posture of the user based on the rotation angle of the motor 33 detected by the rotation detector 36.

The controller 15 processes signals from one or both of the sensor 38 and the rotation detector 36. The controller 15 outputs a control command including a current command value to the actuator 14 (motor 33) according to the result of the process, i.e., the posture of the user. Based on the control command, the actuator 14 (motor 33) is operated to reel and unreel the belt body 13 as well as temporarily stop reeling or unreeling.

Here, an aspect of the operation of the parts when the controller 15 controls the operation of the actuator 14 (motor 33) using signals from the rotation detector 36 will be described. In a state where the assist device 10 is worn by a user, the motor 33 is controlled by the controller 15 so as to operate (generate a torque) in a direction of reeling the belt body 13 with a smaller force than when generating an assist force and exert a small tensile force on the belt body 13. This prevents the belt body 13 from loosening.

When the user changes his or her posture, e.g., assumes a forward leaning posture from an upright standing posture, a tensile force due to the change in posture is exerted on the belt body 13. In this case, therefore, when the user starts to change his or her posture to a forward leaning posture, the motor 33 is forcibly rotated (the motor 33 idles) by the tensile force of the belt body 13 to reel out the belt body 13, without being powered by the actuator 14. Or when the user starts to change his or her posture to a forward leaning posture, the actuator 14 operates, i.e., drives the motor 33 to rotate and thereby unreel the belt body 13.

Conversely, when the user assumes an upright standing posture from a forward leaning posture, the belt body 13 tries to loosen due to the change in posture. In this case, therefore, when the user starts to change his or her posture to an upright standing posture, to maintain the tensile force acting on the belt body 13, the actuator 14 operates, i.e., drives the motor 33 to rotate and thereby reel the belt body 13.

As has been described, the belt body 13 is reeled or unreeled in response to a change in posture of the user. In this reeling or unreeling, the motor 33 is actively or passively rotated to a predetermined rotation angle. This rotation angle is detected by the rotation detector 36. Thus, the amount of operation of the actuator 14 (motor 33) when reeling or unreeling the belt body 13 in response to a change in posture of the user is detected by the rotation detector 36. Then, the controller 15 acquires the amount of operation of the actuator 14 (the rotation angle of the motor 33) when reeling or unreeling the belt body 13 in response to a change in posture of the user, and based on this amount of operation, obtains a posture parameter showing the posture of the user. Based on this posture parameter, the controller 15 can control the operation of the actuator 14 so as to provide the user with an assist force. In this way, the controller 15 controls the operation of the actuator 14 (motor 33) using signals from the rotation detector 36.

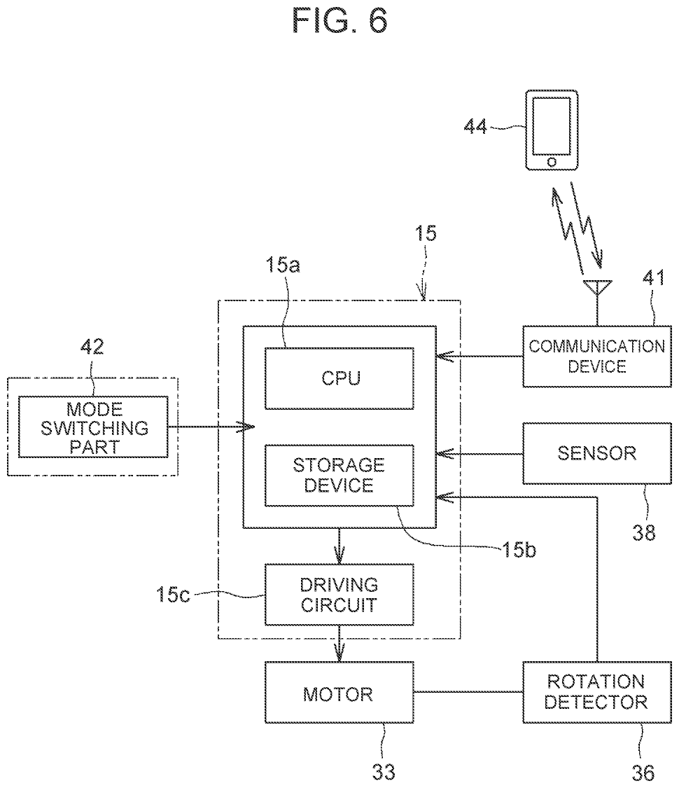

FIG. 6 is a block diagram showing a control configuration of the assist device 10. The rotation detector 36 and the sensor 38 are connected to the controller 15. The controller 15 controls the parts shown in FIG. 6 and acquires outputs from the rotation detector 36 and the sensor 38. Further, the communication device 41 is connected to the controller 15. As described above, the communication device 41 can wirelessly communicate with a user terminal 44. The controller 15 can send and receive information to and from the user terminal 44 by wireless communication through the communication device 41.

The controller 15 is formed by a control unit including a microcomputer, and includes a central processing unit (CPU) 15a and a storage device (storage part) 15b, such as a memory. The central processing unit 15a executes various computation processes based on various programs, various parameters, etc. stored in the storage device 15b. The controller 15 of this disclosure includes a mode switching part 42 as a functional part that is realized by a computation process executed by the central processing unit 15a. The controller 15 further includes a driving circuit (motor driver) 15c that controls the operation of the motor 33. The motor 33 executes a predetermined action as these various functional parts and the driving circuit 15c cooperate with one another. The mode switching part 42 that the controller 15 functionally has will be described later. Assist Force Provided by Assist Device 10

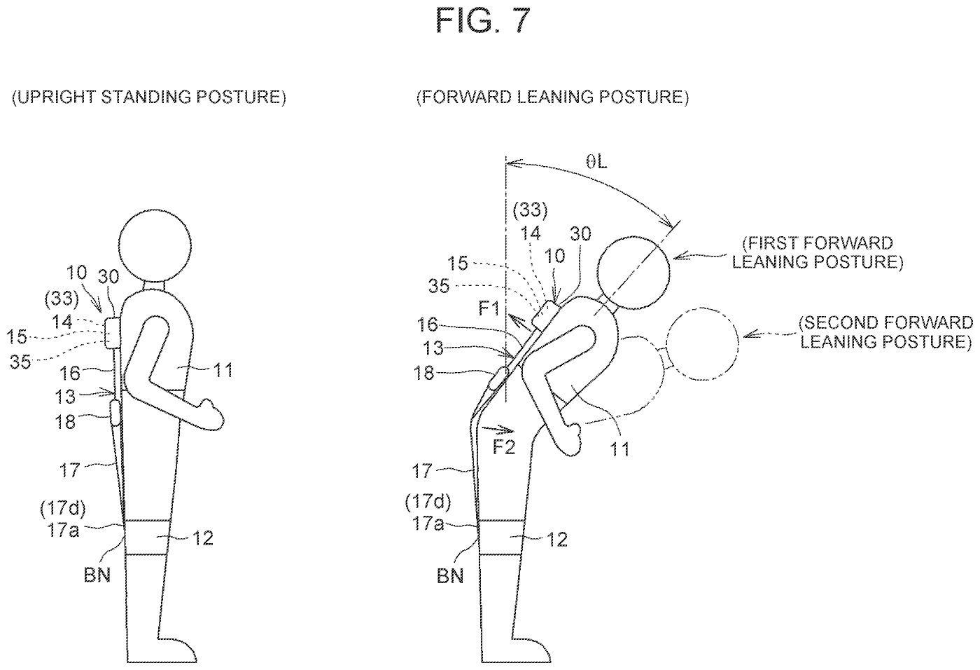

FIG. 7 is a view illustrating a case where the user wearing the assist device 10 changes his or her posture. In response to this change in posture, the assist device 10 can provide an assist force to the user.

When the first belt 16 is reeled onto the driving pulley 35 by the motor 33 of the actuator 14, the coupling member 18 pulls up the second belt 17 toward the actuator 14, i.e., toward an upper side. Both end parts 17a, 17d of the second belt 17 are attached to the left and right second body-worn units 12. The second body-worn units 12 are fixed on the knees BN. Therefore, when the first belt 16 is reeled onto the driving pulley 35, a tensile force is exerted on the first belt 16 and the second belt 17. This tensile force acts as an assist force for the user.

A case where the user assumes a forward leaning posture from an upright standing posture will be described. When the user starts to change his or her posture to a forward leaning posture, the actuator 14 unreels the belt body 13. Or the belt body 13 is reeled out without being powered by the actuator 14. Thus, the user can effortlessly assume a forward leaning posture. When the forward leaning angle of the upper body of the user relative to a vertical line reaches θL and the user stops at this forward leaning angle θL, unreeling (reeling out) of the belt body 13 is stopped. Start and stop of a change in posture can be detected by the rotation detector 36 or the sensor 38.

When the user starts to change his or her posture in a direction from a forward leaning posture to an upright standing posture, the actuator 14 reels the belt body 13. Thus, a tensile force is exerted on the belt body 13. Due to this tensile force, an acting force F1 directed toward the back side is generated in the first body-worn unit 11. This means that the acting force F1 in the direction of raising the upper body of the user in the forward leaning posture is generated. At the same time, an acting force F2 that pushes the left hip and the right hip of the user toward the front side is generated in the second belt 17 due to the tensile force. Thus, the user can easily return from a forward leaning posture to an upright standing posture.

Also when the user maintains a forward leaning posture, the assist device 10 of this disclosure allows the user to easily maintain that posture. Specifically, as shown in the right view of FIG. 7, when the user assumes a first forward leaning posture, the operation of the actuator 14 is stopped to prevent unreeling of the belt body 13. Even when the user tries to assume a posture of leaning further forward (second forward leaning posture), the user cannot assume the second forward leaning posture due to the tensile force of the belt body 13 connecting the first body-worn unit 11 and the second body-worn units 12 to each other. In other words, the assist device 10 tries to keep the user in the first forward leaning posture. This makes it easy for the user to maintain the first forward leaning posture. As a result, the physical burden on the user is reduced, for example, when the user maintains the first forward leaning posture for a long time to perform a task.

Mode Switching Process

As described above, the controller 15 functionally has the mode switching part 42. The mode switching part 42 has a function of executing a mode switching process of switching the control mode of the motor 33 included in the actuator 14. The controller 15 has a function of executing either a normal mode or a restriction mode as the control mode of the motor 33. The normal mode is a mode in which the motor 33 is controlled by a current command value, without restriction on the maximum rotation speed of the motor 33. The restriction mode is a mode in which the motor 33 is controlled while the maximum rotation speed of the motor 33 is restricted to a predetermined rotation speed. In the restriction mode, the maximum rotation speed of the motor 33 is restricted to a predetermined rotation speed.

In the normal mode, the controller 15 performs current control on the motor 33 according to a current command value that is generated according to the posture of the user. In this case, the controller 15 controls the motor 33 according to the current command value, regardless of the rotation speed of the motor 33. On the other hand, in the restriction mode, the controller 15 controls the motor 33 based on the current command value generated according to the posture of the user and on the rotation speed of the motor 33. In this case, the controller 15 performs current control on the motor 33 while controlling the rotation speed of the motor 33 so as not to exceed the predetermined rotation speed. Thus, in the normal mode in which the maximum rotation speed of the motor 33 is not restricted, the motor 33 is controlled so as to operate within a rotation speed range higher than the predetermined rotation speed to which the rotation speed is restricted in the restriction mode.

By executing the mode switching process, the mode switching part 42 switches the control mode of the motor 33 being controlled by the controller 15 to either the restriction mode or the normal mode. The mode switching part 42 executes the mode switching process according to a command given from the outside. This command is sent from the user terminal 44. The user terminal 44, for example, displays a switch for receiving an input operation by the user on a touch-panel etc. of the user terminal 44. The user terminal 44 displays a switch for selecting the restriction mode and a switch for selecting the normal mode. Upon receiving the user's operation by the switch for selecting the restriction mode, the user terminal 44 sends a restriction mode command that is a command ordering the control mode to be switched to the restriction mode to the assist device 10. Upon receiving the user's operation by the switch for selecting the normal mode, the user terminal 44 sends a normal mode command that is a command ordering the control mode to be switched to the normal mode to the assist device 10 by wireless communication.

The assist device 10 receives a restriction mode command and a normal mode command from the user terminal 44 by the communication device 41. The commands received by the communication device 41 are given to the mode switching part 42 of the controller 15.

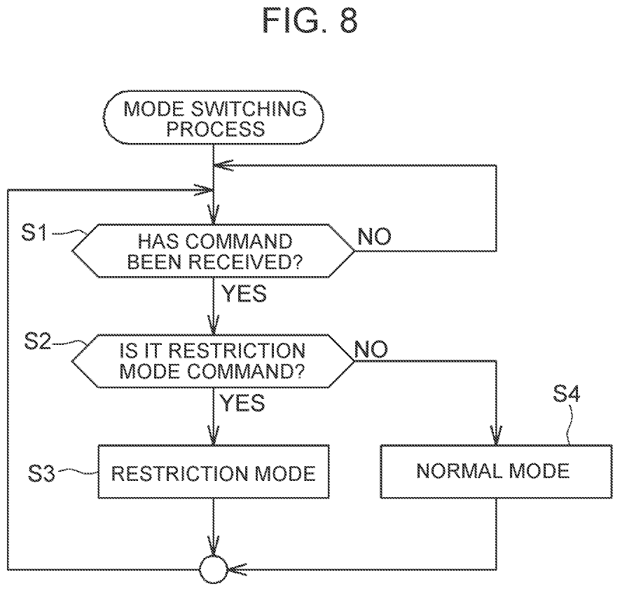

FIG. 8 is a flowchart showing one example of the mode switching process. As shown in FIG. 8, the mode switching part 42 determines as needed whether a command from the user terminal 44 has been received (step S1), and waits for a command. Upon receiving a command, the mode switching part 42 determines whether the received command is a restriction mode command (step S2). When the received command is a restriction mode command, the mode switching part 42 switches the control mode from the normal mode to the restriction mode (step S3), and returns to step S1. When the received command is not a restriction mode command (when it is a normal mode command), the mode switching part 42 switches the control mode from the restriction mode to the normal mode (step S4), and returns to step S1. In this way, the user can switch the control mode of the assist device 10 to either the restriction mode or the normal mode by operating the user terminal 44.

According to the above-described configuration, in the restriction mode, the maximum rotation speed of the motor 33 is restricted to the predetermined rotation speed lower than that in the normal mode, and therefore noise made by the motor 33 as it rotates can be reduced compared with that in the normal mode. Thus, the noise of the motor can be reduced by switching the control mode of the motor 33. In this case, the predetermined rotation speed is set to a rotation speed at which noise made by the motor 33 as it rotates is reduced to such an extent as not to cause discomfort to people around the assist device 10.

In this embodiment, the assist device 10 includes the communication device 41 as an input part that receives commands (a restriction mode command and a normal mode command) as external inputs from the user terminal 44. Thus, the user of the assist device 10 can cause the mode switching part 42 to execute the mode switching process by operating the user terminal 44 and giving a command to the communication device 41. Therefore, the user can reduce the noise of the motor 33 as necessary.

### OTHER EMBODIMENTS

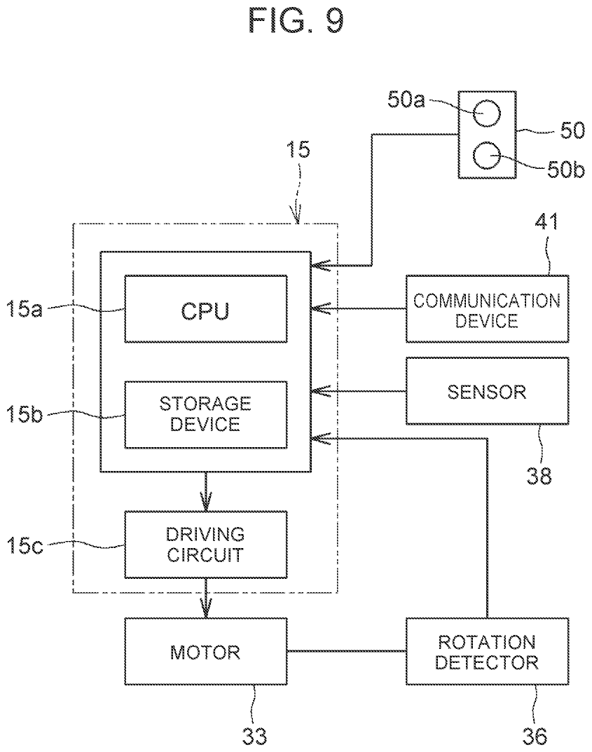

FIG. 9 is a block diagram showing a configuration around a controller according to another embodiment. In the above embodiment, the case where commands (a restriction mode command and a normal mode command) as external inputs are received by the communication device 41 has been illustrated. As shown in FIG. 9, the assist device 10 of this embodiment further includes a switch 50 for receiving an operation input by the user as an external input. The switch 50 is connected to the controller 15 by wire. The switch 50 has a first button 50a for selecting the restriction mode and a second button 50b for selecting the normal mode. The buttons 50a, 50b can receive a press-down operation by the user. When one of the buttons 50a, 50b is pressed down, the controller 15 switches the control mode to a mode corresponding to that one button that has been pressed down.

Thus, the assist device 10 of this embodiment can receive an operation input by the user by means of the switch 50 and cause the controller 15 to execute the mode switching process. While the case where the switch 50 is connected to the controller 15 by wire is shown in FIG. 9, the switch 50 may instead be connected to the controller 15 by wireless communication. When the switch 50 is connected to the controller 15 by wireless communication, the controller 15 may perform wireless communication with the switch 50 by the communication device 41.

Further, the controller 15 may execute the mode switching process according to the current time clocked by an internal clock provided in the controller 15. Usually, it is at night that the noise of the motor 33 is problematic. In this configuration, the control mode of the motor 33 can be switched to the restriction mode during the nighttime (e.g., from seven p.m. to seven a.m.) and the control mode of the motor 33 can be switched to the normal mode at other times of the day than the nighttime. Thus, the noise of the motor 33 at night can be reduced without the user having to make any operation input into the assist device 10. Further, the controller 15 may output an alarm, such as an alert sound, to prompt the user to manually switch the control mode when a preset time has come.

Others

The embodiments disclosed this time are in every respect illustrative and not restrictive. For example, in the above embodiments, the case where the control mode is switched to either the normal mode or the restriction mode has been illustrated. However, the restriction mode may include a plurality of restriction modes different from one another in the predetermined rotation speed that is the maximum rotation speed. In this case, finer adjustments can be made in reducing the noise of the motor 33, and thus adjustments can be made so as to achieve a balance between the operation state of the assist device 10 and the noise reducing effect.

The scope of the right for the present disclosure is not limited to the above embodiments but includes all changes within the scope equivalent to the configuration described in the claims.

What is claimed is:

1. An assist device comprising:
a first body-worn unit that is worn on at least one of the shoulders and the chest of a user;
a second body-worn unit that is worn on one of left and right legs and a waist of the user;
a belt body that is provided along a back side of the user, across the first body-worn unit and the second body-worn unit;
an actuator that is provided in one of the first body-worn unit and the second body-worn unit and configured to reel and unreel a part of the belt body; and
a controller configured to control the actuator, wherein:
the actuator includes a pulley around which a part of the belt body is wound, and a motor that causes the pulley to reel and unreel a part of the belt body; and
the controller is configured to execute a mode switching process of switching a control mode of the motor to one of a restriction mode in which a maximum rotation speed of the motor is restricted to a predetermined rotation speed and a normal mode in which the motor is operated within a rotation speed range higher than the predetermined rotation speed.

2. The assist device according to claim 1, wherein the controller is configured to execute the mode switching process according to a current time.

3. The assist device according to claim 1, further comprising an input part configured to receive an external input, wherein the controller is configured to execute the mode switching process based on the external input.
